(19) United States
(12) Patent Application Publication
(10) Pub. No.: US 2023/0190762 A1
(43) Pub. Date: Jun. 22, 2023

(54) STABLE VARENICLINE DOSAGE FORMS

(71) Applicant: Handa Pharma, Inc., San Jose, CA (US)

(72) Inventors: Michael He, San Jose, CA (US); Zhang Yu, Hangzhou (CN); Li Qiao, San Jose, CA (US); Stephen Cary, San Jose, CA (US)

(73) Assignee: Handa Pharma, Inc., San Jose, CA (US)

(21) Appl. No.: 18/168,310

(22) Filed: Feb. 13, 2023

Related U.S. Application Data
(63) Continuation-in-part of application No. PCT/US2022/034181, filed on Jun. 20, 2022.
(60) Provisional application No. 63/214,916, filed on Jun. 25, 2021.

(51) Int. Cl. A61K 31/55; A61K 9/20

(52) U.S. Cl. CPC A61K 31/55; A61K 9/2077

(57) ABSTRACT

The present invention relates to a stable varenicline solid oral dosage form.

This application is a continuation-in-part of International Patent Application Number PCT/US2022/034181, filed on Jun. 20, 2022, which claims the benefits of U.S. Provisional Patent Application Ser. No. 63/214,916, filed Jun. 25, 2021, each of which is herein incorporated by reference in its entirety.

### FIELD OF THE INVENTION

The present invention relates to pharmaceutical dosage forms and methods for preparing pharmaceutical dosage forms containing varenicline and pharmaceutically acceptable salt thereof, such as the tartrate salt. The pharmaceutical dosage forms of the present invention should be stable upon storage. Embodiments of the present invention should contain less than 50 ppm, preferably less than 25 ppm, and more preferably less than 20 ppm, of any varenicline nitroso impurity.

### BACKGROUND

Varenicline and pharmaceutically acceptable salts thereof are described in U.S. Pat. Nos. 6,410,550; 6,890,927; 7,265,119 which are incorporated herein by reference.

Varenicline is a nicotinic receptor partial agonist. The tartrate salt of varenicline has been approved by the U.S. Food and Drug Administration (FDA) for use as an aid in smoking cessation treatments and is sold under the tradename CHANTIX®. CHANTIX® is a tablet for oral administration comprising varenicline tartrate, microcrystalline cellulose, dibasic calcium phosphate, croscarmellose sodium, silicon dioxide and magnesium stearate. The CHANTIX® tablet is coated with OPADRY® WHITE OR BLUE and an outer most coating of OPADRY® CLEAR. The varenicline tartrate in the CHANTIX® may degrade over time and form nitroso compounds. The nitroso compounds may also be by products of the varenicline synthesis or dosage form manufacture.

Oral dosage forms containing varenicline are described in U.S. Patent Application No. 2004/0235850 and International Patent Application No. WO 2004/103372 which are incorporated herein by reference.

The present invention is a stable varenicline solid oral dosage form and a method for making the stable varenicline solid oral dosage form wherein the varenicline solid oral dosage form that contains less than 50 ppm, preferably less than 25 ppm, and more preferably less than 20 ppm, of any varenicline nitroso impurity including N-nitroso or nitrosamine impurities (R¹—N(—R²)—N═O).

### SUMMARY OF THE INVENTION

The present invention is a stable varenicline solid oral dosage form and a method for making a stable varenicline solid oral dosage form wherein the stable varenicline solid oral dosage form that contains less than 50 ppm, less than 45 ppm, less than 40 ppm, less than 35 ppm, less than 30 ppm, less than 25 ppm, less than 20 ppm, less than 18.5 ppm, less than 15 ppm, less than 12.5 ppm, less than 10 ppm, less than 9 ppm, less than 8 ppm, less than 7 ppm, less than 6 ppm, less than 5 ppm, less than 4 ppm, less than 3 ppm, less than 2 ppm and less than 1 ppm of any varenicline nitroso compound.

In certain embodiments, the stable varenicline solid oral dosage form is a tablet wherein the tablet is prepared by dry granulating varenicline with pharmaceutically acceptable excipients such as lubricants, fillers, binders, disintegrants, glidants, solubilizing agents, flavoring agents, pH adjusting agents, antioxidants, chelating agents, or mixtures of the foregoing, compressing the varenicline granules into a tablet, coating the tablet with a water soluble protective coating material and optionally coating the protective coating with a further cosmetic coating. The coated tablets may be placed in a conventional pharmaceutical package such as a polyethylene or polypropylene bottle with or without a desiccant, and the bottle is sealed. The coated tablets may also be placed in conventional blister packs. In certain embodiments, the coated tablets may be placed in blister packs with high-barrier blister films, such as Aclar® (poly-chloro-tri-fluoro ethylene (“PCTFE”)).

In certain embodiments, the stable varenicline solid oral dosage forms are prepared by first mixing or blending the varenicline or pharmaceutically acceptable salt thereof with a pharmaceutically acceptable excipient, such as a binder, filler or diluent, with a low moisture content, i.e., a moisture content of less than 0.5%, 0.4%, 0.3%, 0.2%, or 0.1%. Examples of low moisture content such excipients include but are not limited to anhydrous lactose and calcium phosphate. The weight ratio of the varenicline or pharmaceutically acceptable salt thereof to the low moisture excipient is at least 1:2 or greater such as 1:3, 1:4, 1:5; 1:6: 1:7; 1:8, 1:9, 1:10 or greater. In certain embodiments, the low moisture content excipients should be porous or irregular shaped and larger than the particle size of the varenicline or pharmaceutically acceptable salt thereof so that the varenicline or pharmaceutically acceptable salt thereof may enter the pores or crevices of the low moisture content excipient during the blend step. This two component mixing of the varenicline and low moisture excipient(s) should provide a more uniform distribution of the varenicline within the low moisture excipient(s) and/or absorption or adhesion of the varenicline onto the surface of the low moisture excipient(s) in the dosage form and prior to the blending and/or granulation of the varenicline and low moisture excipient mixture with other pharmaceutically acceptable excipients.

The blend of the varenicline or pharmaceutically acceptable salt thereof and low moisture excipient(s) may be dry granulated or blended with additional excipients such as lubricants, fillers, binders, disintegrants, glidants, solubilizing agents, flavoring agents, pH adjusting agents, antioxidants, chelating agents, or mixtures of the foregoing and dry granulated. The granules may be compressed into tablets or the granules may be further blended with additional extra granular excipients such as lubricants, fillers, binders, disintegrants, glidants, solubilizing agents, flavoring agents, pH adjusting agents, antioxidants, chelating agents, or mixtures of the foregoing and compressed into tablets.

The dry granulation method may be roller compaction or slugging.

In certain embodiments, the bulk or raw varenicline or pharmaceutically acceptable salt thereof employed in the preparation of the dosage forms described herein should have an initial total nitrosamine content of 6 ppm or less, 5 ppm or less, 4 ppm or less, 3 ppm or less, 2 ppm or less or 1 ppm or less. The initial total nitrosamine content of the bulk or raw varenicline or pharmaceutically acceptable salt thereof should be determined by any acceptable method at least 30 days, at least 20 days, at least 14 days, at least 10 days, at least 7 days, at least 5 days, at least 4 days, at least 3 days, at least 2 days, at least 1 day (24 hours) prior to begin the manufacturing process, and preferably prior to the initial blending, mixing or granulating of the bulk or raw varenicline or pharmaceutically acceptable salt thereof with one or more excipients.

In certain embodiments, the total manufacturing or tablet processing time should be less than 60 days, preferably less than 30 days and more preferably less than 15 days. In certain aspects, the total manufacturing or tablet processing time begins with the first combination, i.e. mixing or blending, of the drug and at least one excipient and ends with the compressing of the drug and at least one excipient into the tablet of the present invention. The processing time may also include the initial weighing of the drug and at least one excipient, the coating of the compressed tablet, the packaging of the tablets and a combination thereof.

In certain embodiments, the final dosage forms should be packaged in a bottle or blister package. If the packaging is a bottle, the bottle should protect the dosage from light and moisture and preferably contain a desiccant. If the packaging is a blister package, the blister may be formed from PVC or PCTFE with an aluminum backing. One or more of the blister cards may be placed is a bag, preferable a foil bag along with a desiccant.

The present invention further includes methods of using the tablets and kits comprising the tablets and packaging as described herein.

### DETAILED DESCRIPTION OF THE INVENTION

Before the present invention is further described, it is to be understood that this invention is not limited to the particular embodiments described. It is also to be understood that the terminology used herein is for the purpose of describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “normal storage conditions” refers to storage at room temperature, approximately 25° C. and approximately 60% relative humidity for at least three months, preferably at least six months, and most preferably at least one year. The solid dosage form in accordance with the present invention should be stored in pharmaceutically acceptable containers such as glass bottles, plastic bottles, metal foil pouch, or blister packaging with or without a desiccant.

As used herein, the term “accelerated storage conditions” refers to storage at approximately 40° C. and approximately 75% relative humidity for at least two weeks or longer, one month or longer, two months or longer, three months or longer, four months or longer, five months or longer, or six months or longer. The solid dosage form in accordance with the present invention should be stored in pharmaceutically acceptable containers such as glass bottles, plastic bottles, metal foil pouch, or blister packaging with or without a desiccant.

As used herein the nitroso varenicline compounds included but not limited to:

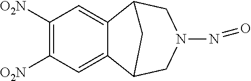 [SMILES: O=NN1CC2CC(C1)c1cc([N+](=O)[O-])c([N+](=O)[O-])cc12]

4,5-Dinitro-10-nitroso-10-azatricyclo[6.3.1.02, 7]dodeca-2,4,6-triene (Nitro-P06)

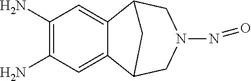 [SMILES: Nc1cc2c(cc1N)C1CC2CN(N=O)C1]

3-Nitroso-2,3,4,5-tetrahydro-1H-1,5-methanobenzo[d]azepine-7,8-diamine (Nitro-P07) and

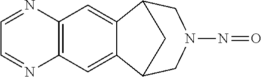 [SMILES: O=NN1CC2CC(C1)c1cc3nccnc3cc12]

8-nitroso-7,8,9,10-tetrahydro-6H-6,10-methanoazepino[4,5-g]quinoxaline (Nitro-P08)

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The varenicline, preferably varenicline tartrate employed in the preparation of the dosage forms of the present invention can have a range of particle sizes. In certain embodiments, the varenicline tartrate exhibit a particle size (D90) of a range from 350 microns to 20 microns, preferably from 300 microns to 50 microns and most preferably from 250 microns to 100 microns. In certain embodiments, the varenicline should not be micronized and exhibit a particle size (D90) of larger than 50 microns, larger than 75 microns, larger than 100 microns, larger than 125 microns, larger than 150 microns, larger than 175 microns or larger than 200 microns. In certain embodiments, the varenicline should be micronized and exhibit a particle size (D90) of less than 300 microns, less than 200 microns, less than 100 microns, less than 50 microns, preferably less than 35 microns and most preferably less than 20 microns. In certain embodiments, the varenicline tartrate exhibit a particle size (D50) of a range from 50 microns to 1 microns, preferably from 40 microns to 1 microns and most preferably from 30 microns to 5 microns. In certain embodiments, the varenicline should exhibit a particle size (D50) of larger than 1 microns, larger than 5 microns, larger than 10 microns, larger than 15 microns, larger than 20 microns or larger than 25 microns. In certain embodiments, the varenicline should exhibit a particle size (D50) of less than 30 microns, less than 25 microns, less than 20 microns, preferably less than 15 microns and most preferably less than 10 microns. In certain embodiments, the varenicline tartrate exhibit a particle size of volume median diameter (VIVID) of a range from 150 microns to 20 microns, preferably from 100 microns to 25 microns and most preferably from 80 microns to 35 microns. In certain embodiments, the varenicline should exhibit a VIVID particle size of larger than 20 microns, larger than 25 microns, larger than 30 microns, larger than 35 microns or larger than 40 microns. In certain embodiments, the varenicline should exhibit a VIVID particle size of less than 60 microns, less than 55 microns, less than 50 microns, preferably less than 40 microns and most preferably less than 30 microns.

In certain embodiments, the varenicline tartrate exhibit a particle size (D90) of a range from 50 microns to 1 microns, preferably from 30 microns to 2 microns and most preferably from 20 microns to 3 microns. In certain embodiments, the varenicline should not be micronized and exhibit a particle size (D90) of larger than 1 microns, larger than 2 microns, larger than 3 microns or larger than 5 microns. In certain embodiments, the varenicline should be micronized and exhibit a particle size (D90) of less than 50 microns, less than 40 microns, less than 30 microns, preferably less than 20 microns and most preferably less than 15 microns. In certain embodiments, the varenicline tartrate exhibit a particle size (D50) of a range from 30 microns to 1 microns, preferably from 20 microns to 1 microns and most preferably from 10 microns to 2 microns. In certain embodiments, the varenicline should exhibit a particle size (D50) of larger than 1 micron, larger than 2 microns, larger than 3 microns, larger than 4 microns or larger than 5 microns. In certain embodiments, the varenicline should exhibit a particle size (D50) of less than 30 microns, less than 20 microns, preferably less than 15 microns and most preferably less than 10 microns.

The particle size may be determined by any method commonly employed in the pharmaceutical arts, some of which are described in Remington, The Science and Practice of Pharmacy 21^{st} ed. (2005) pp. 706-711 which is incorporated herein by reference.

The bulk or raw varenicline, preferably varenicline tartrate employed in the preparation of the dosage forms of the present invention should have a low initial total nitrosamine content. The low initial total nitrosamine content should be 7 ppm or less, 6 ppm or less, 5 ppm or less, 4 ppm or less, 3 ppm or less, 2 ppm or less, 1 ppm or less. In certain embodiments, the low initial total nitrosamine content can range from about 0 to about 5 ppm, about 0.25 ppm to about 4.5 ppm, about 0.5 ppm to about 4 ppm, about 0.75 ppm to about 3.75 ppm, about 1 ppm to about 3.5 ppm or any value within the aforementioned ranges. The foregoing nitrosamine values and the nitrosamine values described elsewhere herein are based on the varenicline free base content of the composition unless stated otherwise. The amounts will vary slightly if different salts or if the free base is used. Typically, the level is based on the acceptable intake (AI) limit, wherein the ppm is calculated based on a drug's maximum daily dose (MDD) as reflected in the drug label. More specifically, ppm=AI (ng)/MDD (mg). A more detailed discussion for determining acceptable nitrosamine levels in bulk drug product and finished dosage forms can be found in the U.S. FDA Guidance for Industry Document, entitled “Control of Nitrosamine Impurities in Human Drugs” published February 2021, the contents of which are incorporated herein by reference.

The initial total nitrosamine content may be determined by any method commonly used in the pharmaceutical industry and preferably may be determined by LC-MS (Liquid Chromatography Mass Spectrometry), HPLC (High Performance Liquid Chromatography) or UPLC (Ultra Performance Liquid Chromatography) as described below. The initial total nitrosamine content should be determined prior to manufacturing the dosage form, preferably prior to the initial blending, mixing or granulating of the bulk or raw varenicline or pharmaceutically acceptable salt thereof with one or more excipients. In certain embodiments, the initial total nitrosamine content is determined about 30 days or less, about 25 days or less, about 20 days or less, about 15 days or less, about 10 days or less, about 9 days or less, about 8 days or less, about 7 days or less, about 6 days or less, about 5 days or less, about 4 days or less, about 3 days or less, about 2 days or less or about 1 day or less prior to the initial blending, mixing or granulating of the bulk or raw varenicline or pharmaceutically acceptable salt thereof with one or more excipients.

In certain embodiments the initial total nitrosamine content is determined about 0.5 to about 14 days, about 0.75 to about 10 days, about 1 to about 7 days or any value within the aforementioned ranges.

Once the initial total nitrosamine content is determined, the bulk or raw varenicline or pharmaceutically acceptable salt thereof, may be stored under conditions that protected it from light, moisture and/or oxygen until it is to be blended, mixed or granulated with one or more excipients. The storage may comprise one or more plastic bags, preferably at least two or more plastic bags with suitable desiccant between the bags and the bags stored in a container that prevents exposure to light such as a cardboard drum. The storage may also comprise purging the plastic bags or storage container with an inert gas such as nitrogen to remove or reduce the amount of oxygen in the storage system.

The stable varenicline solid dosage forms of the present invention should comprise a therapeutically effective amount of varenicline or a pharmaceutically acceptable salt, conjugate or complex thereof. The therapeutically effective amount can be easily determined from a review of the available literature and can range from about 0.1 mg to about 5 mg, preferably about 0.25 mg to about 2.0 mg, and most preferably about 0.5 mg to about 1.0 mg.

The stable varenicline solid dosage forms of the present invention may comprise about 0.25 wt % to about 5 wt %, preferably about 0.5 wt % to about 2.5 wt %, and preferably about 0.70 wt % to about 1.0 wt % of varenicline or a pharmaceutically acceptable salt thereof and about 10 wt % to about 99 wt % of one or more low moisture excipients, preferably about 15 wt % to about 97 wt %, and most preferably about 20 wt % to about 95 wt %.

The pharmaceutically acceptable solid dosage form of the present invention may further comprise conventional pharmaceutically acceptable excipients such as lubricants, fillers, binders, disintegrants, glidants, solubilizing agents, flavoring agents, pH adjusting agents, antioxidants or mixtures of the foregoing. The amount of these excipients present in the solid dosage forms will vary depending upon the specific and desired properties of the solid dosage form. Ranges and amounts of these excipients are known and reported in the literature.

Examples of lubricants that may be employed in the solid dosage form of the present invention include magnesium stearate, sodium stearyl fumarate, stearic acid, glyceryl behenate, polyethylene glycols (preferably wherein the polyethylene glycol has a molecular weight of 6000 or more), polyoxyethylene stearate, magnesium lauryl sulfate, sodium oleate, and mixtures thereof. The lubricants may be present in an amount ranging from about 0.1 wt % to about 10 wt % based on the total weight of the dosage form, preferably about 0.2 wt % to about 7 wt %, and most preferably about 0.5 wt % to about 5 wt %.

Examples of fillers that may be employed in the solid dosage form of the present invention include dibasic calcium phosphate (anhydrous), microcrystalline cellulose, calcium carbonate, magnesium carbonate, calcium sulfate, powdered cellulose, silicified microcrystalline cellulose, magnesium carbonate, magnesium oxide, starch, lactose anhydrous, mannitol and mixtures thereof.

Examples of binders that may be employed in the solid dosage form of the present invention include acacia, povidone, hypromellose, hydroxypropyl cellulose, hydroxyethyl cellulose, polyethylene oxide, polymethacrylates, methyl cellulose, ethyl cellulose, pregelatinized starch, microcrystalline cellulose, gelatin, tragacanth, zein, or mixtures thereof. Preferably, the binder is selected from povidone, hypromellose, hydroxypropyl cellulose, hydroxyethyl cellulose, polymethacrylates, methyl cellulose, gelatin and ethyl cellulose, or mixtures thereof. Especially preferred binders include water soluble binders such as povidone, hypromellose, hydroxypropyl cellulose, gelatin and mixtures thereof If the binder is a polymeric binder, it is preferred that the binder have a low molecular weight and/or exhibit a viscosity of less than 200 mPa·s, preferably less than 100 mPa·s, and most preferably less than 50 mPa·s when tested at a concentration of 2% (w/v) aqueous preparation at 20° C.

Examples of disintegrants that may be employed in the solid dosage form of the present invention include croscarmellose sodium, starch, crospovidone, sodium starch glycolate, alginic acid, calcium carboxymethylcellulose, sodium carboxymethylcellulose, calcium carboxymethylcellulose, powdered cellulose, chitosan, guar gum, magnesium aluminum silicate, methylcellulose, sodium alginate, and mixtures thereof.

Examples of glidants that may be employed in the solid dosage form of the present invention include colloidal silicon dioxide, corn starch, talc and mixtures thereof

One or more solubilizing agents may be employed in the dosage forms of the present invention. Examples of solubilizing agents that may be used in various embodiments of the present invention include but are not limited to cyclodextrins, surfactants (sometimes referred to as wetting agents) and mixtures thereof.

Examples of pH adjusting agents that may be employed in the solid dosage forms of the present invention include pharmaceutically acceptable acids or bases which may be present to adjust the pH of intermediate compositions leading up to the final solid dosage form and to adjust the pH of the drug environment of final solid dosage form to a desired or optimum pH range. Representative examples of pharmaceutically acceptable acids that may be used include, but are not limited to, acetic acid, citric acid, fumaric acid, hydrochloric acid, malic acid, nitric acid, phosphoric acid, propionic acid, sulfuric acid, tartaric acid, and mixtures thereof. Representative examples of pharmaceutically acceptable bases that may be used include but are not limited to ammonia, ammonium carbonate, diethanolamine, potassium hydroxide, sodium bicarbonate, sodium carbonate, sodium hydroxide, trolamine, and mixtures thereof.

Examples of antioxidants that may be employed in the solid dosage forms of the present invention include ascorbic acid, ascorbyl palmitate, butylated hydroxyanisole, butylated hydroxytoluene, hypophosphorous acid, monothioglycerol, potassium metabisulfate, propyl gallate, sodium bisulfite, sodium formaldehyde sulfoxylate, sodium metabisulfate, sodium sulfate, sodium thiosulfate, sodium dioxide, tocopherol, and mixtures thereof. The antioxidant may be present in the dosage forms of the present invention in an amount from about 0.01 wt % to about 20 wt % based upon the total weight of the dosage form, preferably from about 0.1 wt % to about 10 wt %, and most preferably from about 0.5 wt % to about 5 wt %.

When the dosage form is a tablet, the tablet core comprising a compressed blend of the varenicline, low moisture excipient(s) and other pharmaceutically acceptable excipients can be coated with one or more coatings, preferably at least two coatings. The coating may be water soluble or water dispersible and rapidly dissolve or disintegrate when the coated tablet is placed in 500 to 900 ml of an aqueous solution such as simulated intestinal fluid or simulated gastric fluid. Rapidly dissolving or disintegrating means the coating or coatings dissolve or disintegrate in less than 15 minutes, less than 14, 13, 12, 11, 10, 9, 8, 7, 6, 5, 4, 3, 2, or 1 minute when places in the aqueous environment. In certain embodiments the coating(s) comprises a polymer such as hydroxypropyl methylcellulose, hydroxypropyl cellulose, polyvinylalcohol, polyvinylpyrrolidone or combinations thereof. The polymer exhibits a viscosity of less than 50 cps, preferably less than 45, 40, 35, 30, 25, 20, 15, 10 or 5 cps when a 2% aqueous solution is prepared and tested at ambient conditions. In certain embodiments the polymer employed in the coating is water soluble. In certain embodiments the coating should be applied to the tablet core in an amount ranging from 0.5% to about 10%, preferably about 1% to about 8% and most preferably about 2.5% to about 7.5% wherein the weight percent of the coating is based on the percent weight gain of the core tablet. For example if the core weighs 100 mg, a 2.5% coating means 2.5 mg of coating is applied to the core.

In certain embodiments, at least two coats are applied to the tablet preferably the first coat comprises a low viscosity polyvinyl alcohol or hydroxpropyl methylcellulose (aka hypromellose) with or without a pigment and with or without polyethylene glycol and the second coat comprises a low viscosity polyvinyl alcohol or hydroxpropyl methylcellulose with or without a pigment and with or without polyethylene glycol. In certain aspects of this embodiment, the first coating (as protective coat) applied to the tablet core comprises a low viscosity polyvinyl alcohol or hydroxpropyl methylcellulose without a pigment and the second coating (as color coat) applied to the first protective coating comprises a low viscosity polyvinyl alcohol or hydroxpropyl methylcellulose with a pigment. The weight ratio of the first coat to the second coat should range from about 1:4 to about 4:1, preferably about 1:3 to about 3:1, more preferably about 1:2 to about 2:1 and most preferably about 1:1 to about 1:2 or 1:2 to about 1:4. In certain embodiments, the weight ratio of the coat without a pigment to the coat with a pigment should range from about 1:4 to about 4:1, preferably about 1:3 to about 3:1, more preferably about 1:2 to about 2:1 and most preferably about 1:1 to about 1:2 or 1:2 to about 1:4. Alternatively, the first coat may comprise about 20% to about 70%, about 25% to about 60%, about 30% to about 50% of the total coating weight (i.e. total weight of the first and second coating). In certain embodiments, the coat without a pigment may comprise about 20% to about 70%, about 25% to about 60%, about 30% to about 50% of the total coating weight. In certain aspects of this embodiment, the first coat should exhibit water vapor transmission rate of about 100 to about 500, about 100 to about 400, about 100 to about 300 or about 100 to about 200 μg/day/mm² when the coat is applied to the tablet core at a 4% weight gain the coated tablet is stored at 40° C. and 75% relative humidity.

In certain embodiments the dosage form of the present invention, preferably the tablet of the present invention and most preferably the coated tablet of the present invention should exhibit a moisture content of about 1.0% to about 8.0%, preferably about 1.5% to about 7.0% and most preferably about 1.7% to about 5.0% when manufactured and packaged for distribution.

Alternatively, the dosage form of the present invention, preferably the tablet of the present invention and most preferably the coated tablet of the present invention should exhibit a moisture content of less than 7.5%, preferably than 7.0%, 6.5%, 6.0%, 5.5%, 5.0%, 4.5%, 4.0%, 3.5%, 3.0% 2.5% or 2.0% when manufactured and packaged for distribution.

The dosage form of the present invention, preferably the tablet of the present invention and most preferably the coated tablet of the present invention should also exhibit a moisture content of about 1.0% to about 8.0%, preferably about 1.5% to about 7.0% and most preferably about 1.7% to about 5.0% when stored in a sealed bottle, preferably a sealed plastic bottle such as a high density polyethylene bottle (with or without a desiccant) or a sealed blister packaging such as a sealed blister card or a foil sealed blister at approximately 25° C. and approximately 60% relative humidity for at least three months, preferably at least six months and most preferably at least one year and/or at approximately 40° C. and approximately 75% relative humidity for one month, two months, or three months. Alternatively, the dosage form of the present invention, preferably the tablet of the present invention and most preferably the seal coated tablet of the present invention should exhibit a moisture content of less than 7.5%, preferably less than 7.0%, 6.5%, 6.0%, 5.5%, 5.0%, 4.5%, 4.0%, 3.5%, 3.0% 2.5% or 2.0% when stored in a sealed bottle, preferably a sealed plastic bottle such as a high density polyethylene bottle (with or without a desiccant) or a sealed blister packaging such as a sealed blister card or a foil sealed blister at approximately 25° C. and approximately 60% relative humidity for at least three months, preferably at least six months and most preferably at least one year and/or at approximately 40° C. and approximately 75% relative humidity for one month, two months, or three months.

The solid dosage forms of the present invention should be stable. More specifically, the solid dosage forms of the present invention will contain less than 50 ppm, less than 45 ppm, less than 40 ppm, less than 35 ppm, less than 30 ppm, less than 25 ppm, less than 20 ppm, less than 18.5 ppm, less than 15 ppm, less than 12.5 ppm, less than 10 ppm, less than 9 ppm, less than 8 ppm, less than 7 ppm, less than 6 ppm, less than 5 ppm, less than 4 ppm, less than 3 ppm, less than 2 ppm and less than 1 ppm of any varenicline nitroso compound when the solid dosage form is stored in a sealed bottle, preferably a sealed plastic bottle such as a high density polyethylene bottle (with or without a desiccant) or a sealed blister packaging such as a sealed blister card or a foil sealed blister, at approximately 25° C. and approximately 60% relative humidity for at least three months, preferably at least six months and most preferably at least one year and/or at approximately 40° C. and approximately 75% relative humidity for one month, two months, or three months.

The solid dosage forms of the present invention should also contain less than 50 ppm, less than 45 ppm, less than 40 ppm, less than 35 ppm, less than 30 ppm, less than 25 ppm, less than 20 ppm, less than 18.5 ppm, less than 15 ppm, less than 12.5 ppm, less than 10 ppm, less than 9 ppm, less than 8 ppm, less than 7 ppm, less than 6 ppm, less than 5 ppm, less than 4 ppm, less than 3 ppm, less than 2 ppm and less than 1 ppm of total varenicline nitroso compounds when the solid dosage form is stored in a sealed bottle, preferably a sealed plastic bottle such as a high density polyethylene bottle (with or without a desiccant) or a sealed blister packaging such as a sealed blister card or a foil sealed blister, stored at approximately 25° C. and approximately 60% relative humidity for at least three months, preferably at least six months, and most preferably at least one year and/or at approximately 40° C. and approximately 75% relative humidity for one month, two months, or three months. The sealed blister packaging may also be further packaged in a bag with or without desiccant. The bag, if employed, may be a plastic bag comprising a single polymeric material or a composite material comprising more than one polymeric materials. The bag may also be made of a foil, such as aluminum foils or a composite comprising at least one foil layer and at least one polymeric layer.

Some embodiments of the present invention will contain less than 20 ppm, less than 18.5 ppm, less than 15 ppm, less than 12.5 ppm, less than 10 ppm, less than 9 ppm, less than 8 ppm, less than 7 ppm, less than 6 ppm, less than 5 ppm, less than 4 ppm, less than 3 ppm, less than 2 ppm and less than 1 ppm of any varenicline nitrosamine compound when the solid dosage form is stored in a sealed bottle, preferably a sealed plastic bottle such as a high density polyethylene bottle (with or without a desiccant) or a sealed blister packaging such as a sealed blister card or a foil sealed blister, at approximately 25° C. and approximately 60% relative humidity for at least three months, preferably at least six months and most preferably at least one year and/or at approximately 40° C. and approximately 75% relative humidity for one month, two months, or three months.

Some embodiments of the present invention should also contain less than 30 ppm, less than 25 ppm, less than 20 ppm, less than 18.5 ppm, less than 15 ppm, less than 12.5 ppm, less than 10 ppm, less than 9 ppm, less than 8 ppm, less than 7 ppm, less than 6 ppm, less than 5 ppm, less than 4 ppm, less than 3 ppm, less than 2 ppm and less than 1 ppm of total varenicline nitrosamine compounds when the solid dosage form is stored in a sealed bottle, preferably a sealed plastic bottle such as a high density polyethylene bottle (with or without a desiccant) or a sealed blister packaging such as a sealed blister card or a foil sealed blister, stored at approximately 25° C. and approximately 60% relative humidity for at least three months, preferably at least six months, and most preferably at least one year and/or at approximately 40° C. and approximately 75% relative humidity for one month, two months, or three months.

In certain embodiments of the present invention, the total manufacturing or tablet processing time should be less than 60 days, preferably less than 30 days and more preferably less than 15 days. In certain aspects of this embodiment, the total manufacturing or tablet processing time is at least 1, 2, 3, 4, 5, 6, 7, 8, 9, 10, 11, 12, 13, 14, or 15 days. The total manufacturing or tablet processing time begins with the first combination of the drug and at least one excipient and ends with the compressing of the drug and at least one excipient into the tablet of the present invention. The first combination may be a mixing, blending or granulation step of the varenicline and at least one pharmaceutically acceptable excipient. In certain aspects of this embodiment, the total manufacturing or tablet processing time may begin when the varenicline is weighed or measured for use in the manufacturing process or alternatively when the varenicline is screened for use in the manufacturing process and prior to the first combination of the varenicline with at least one pharmaceutical acceptable excipient. In a further aspect of this embodiment, the total manufacturing or tablet processing time may also include the time for applying one or more coatings to the compressed tablets, the time required to package the coated or uncoated tablets in the desired commercial packaging, i.e., sealed bottle or blister packaging and a combination thereof.

The tablet manufacturing or tablet processing should preferably be performed in a continuous or substantially continuous process wherein there is no lag or hold time between individual processing steps of greater than 72, 66, 60, 54, 48, 42, 36, 30, 24, 18, 12, or 6 hours. It is preferred that time from the initial weighing or screening of the varenicline to the compression of the varenicline and at least one pharmaceutically acceptable excipient into a table is performed in a continuous or substantially continuous process wherein there is no lag or hold time between individual processing steps of greater than 48, 45, 42, 39, 36, 33, 30, 27, 24, 21, 18, 15, 12, 9, or 6 hours. The individual processing steps include but are not limited to (i) weighing; (ii) screening; (iii) mixing; (iv) blending; (v) granulation; (vi) compression and combinations therefore. It is also understood that more than one of the foregoing steps may be included in the process. For example one, two, three or more screening steps, one, two, three or more mixing steps, one, two, three or more blending steps, and one, two, three or more granulation steps may be included in the total manufacturing or tablet processing. It is also understood that no particular order of the steps is required as long as the time of the processing is followed as outlined above.

In a further aspect of the present invention, if there is a lag between processing steps of greater than 0.5, 1, 1.5, 2, 2.5 or 3 hours, the intermediate compositions formed during a processing step should be protected from light, moisture and/or oxygen as much as possible. For example, if an intermediate composition formed during a step of the manufacturing process will not be used in the next processing step for at least 0.5, 1, 1.5, 2, 2.5, 3, 3.5, 4, 4.5, 5 hours or longer, the intermediate composition will be stored in a sealed container, preferably a glass bottle, a plastic bottle, a stainless steel container, a single, double or multiple plastic bags, that will protect the intermediate composition from exposure to light and ambient moisture and oxygen as possible during the lag time. In addition, the ambient conditions during the manufacturing of the tablets and the individual processing steps should maintain a humidity between about 30% to about 60% and a temperature between about 20° to about 30° C.

In certain embodiments, the tablets of the present invention may be packaged under a nitrogen environment. The tablets may be packed in a sealed glass or plastic bottle, preferably a plastic bottle such as a high density polyethylene bottle with a desiccant. The tablet may also be packaged in a typical blister packaging, preferably an aluminum foil sealed blister. In certain embodiments the blister material may comprise a high moisture barrier film such as PCTFE with a thickness of 35 μm, 40 μm, 45 μm, 50 μm or greater or low moisture barrier film such as PVC with a thickness of 200 μm, 225 μm, 250 μm or greater. In addition to the PCTFE and PVC, other examples of the blister material include poylvinylidene chloride (PVDC); polychlorotrifluoroethylene (PCTFE), polypropylene (PP), polyethylene (PE), cyclic olefin copolymers (COC), metal foils such as aluminum foil or combinations thereof, including multiple layer laminate of the foregoing. The water vapor transmission rate for the blister employed in the present invention may be at least 0.05 g/m²*day or greater, at least 0.08 g/m²*day or greater, at least 0.12 g/m²*day or greater, at least 0.15 g/m²*day or greater, at least 0.30 g/m²*day or greater, at least 0.5 g/m²*day or greater, at least 1.0 g/m²*day or greater, at least 2.0 g/m²*day or greater, at least 2.5 g/m²*day or greater. The lidding material for the blister may be aluminum foil, PE, paper, cardboard of a combination thereof. The lidding material may allow for a push through packaging wherein the dosage form is removed from the blister by pushing it through the lidding or peel away wherein the dosage form is removed from the blister by peeling the lidding away from the blister shell.

Each bottle may comprise 112 tablets or less, 109 tablets or less, 84 tablets or less, 81 tablets or less, 56 tablets or less, 53 tablets or less, 42 tablets or less, 28 tablets or less, 25 tablets or less, 14 tablets or less, 11 tablets or less, 7 tablets or less, 4 tablets or less. Each blister card may comprise 56 tablets or less, 53 tablets or less, 42 tablets or less, 28 tablets or less, 25 tablets or less, 14 tablets or less, 11 tablets or less, 7 tablets or less, 4 tablets or less. One or more of the blister cards may be further packaged in a plastic or metal foil bag with a desiccant. In certain embodiments, the dosage forms, preferably the tablets are packaged with a desiccant. The desiccant may be any type of desiccant commonly used in the pharmaceutical industry including but not limited to silica gel, bauxite, calcium sulfate, calcium chloride, clays, molecular sieves or any combination of the foregoing.

In certain aspects, the desiccant may comprise a silica gel, such as a silica gel bag or canister in amount of at least 0.1 g or more of silica gel per 10 ml volume of container, at least 0.2 g or more of silica gel per 10 ml volume of container, at least 0.3 g or more of silica gel per 10 ml volume of container, at least 0.4 g or more of silica gel per 10 ml volume of container, at least 0.5 g or more of silica gel per 10 ml volume of container, preferably at least 0.75 g or more of silica gel per 10 ml volume of the container, more preferably at least 1 g or more of silica gel per 10 ml volume of the container and most preferably at least 1.5 g or more of silica gel per 10 ml volume of the container.

In certain aspects, the dosage forms, preferably the tablets are packaged with desiccants wherein the desiccants exhibit an Absorption rate (under 25° C.) at least 5% or more, 6% or more, 7% or more, 8% or more under RH20%; at least 15% or more, 17% or more, 19% or more, 21% or more, 23% or more under RH40%; at least 15% or more, 17% or more, 19% or more, 21% or more, 23% or more under RH50%; at least 23% or more, 25% or more, 27% g or more, 29% or more, 31% or more under RH80%; at least 25% or more, 27% or more, 29% or more, 31% or more, 33% or more under RH90%.

In certain aspects, the dosage forms, preferably the tablets are packaged with desiccants wherein the desiccants exhibit an Absorption capacity (under 25° C.) at least 0.01 g or more, 0.02 g or more, 0.03 g or more, 0.04 g or more, 0.05 g or more per 10 ml volume of container under RH20%; at least 0.04 g or more, 0.06 g or more, 0.08 g or more, 0.10 g or more, 0.12 g or more per 10 ml volume of container under RH40%; at least 0.04 g or more, 0.06 g or more, 0.08 g or more, 0.10 g or more, 0.12 g or more per 10 ml volume of container under RH50%; at least 0.06 g or more, 0.09 g or more, 0.12 g or more, 0.15 g or more, 0.17 g or more per 10 ml volume of container under RH80%; at least 0.06 g or more, 0.09 g or more, 0.12 g or more, 0.15 g or more, 0.18 g or more per 10 ml volume of container under RH90%.

If the tablets of the present invention are packaged in a bottle for dispensing by a pharmacist to a patient or provided to a patient in a bottle for administration to the patient, the bottle may include a warning to use, dispense and/or consume the tablets within 60, 55, 50, 45, 40, 35 or 30 days of date the bottle is first opened and if not used, dispensed and/or consumed within the designated period the remaining tablets should be destroyed.

The present invention also includes pharmaceutical kits useful, for example, in aiding in smoking cessation treatments, which include one or more bottles or blister packages containing the tablets described herein. Such kits can further include, if desired, one or more of various conventional pharmaceutical kit components, such as, for example, instructions, either as inserts or as labels, indicating quantities of the tablets to be administered and/or guidelines for administration.

The kit should comprise a combination of a first bottle comprising eleven (11) of the 0.5 mg varenicline tablets as described herein, preferably as described in Example 6 and a second bottle comprising 7-200, preferably 7 to 154 and more preferably 7 to 120 of the 1.0 mg varenicline tablets as described herein, preferably as described in Example 6, and instructions for dosing/administration of the tablets. Preferably the instructions are printed and inform the user to: (i) begin treatment by orally administering one (1) 0.5 mg tablet once a day for the first three days; (ii) on days 4 to 7 of treatment, to orally administer one (1) 0.5 mg tablet twice a day (B.I.D.)

or every twelve hours and (iii) to orally administer one (1) 1.0 mg tablet twice a day (B.I.D.) or every twelve hours on day 8 of treatment until all the 1.0 mg tablets in the second bottle are consumed wherein the eleven (11) 0.5 mg varenicline tablets are obtained from the first bottle in the kit and the 1.0 mg varenicline tablets are obtained from the second bottle in the kit.

In certain embodiments the kit comprises a combination of a first bottle comprising eleven (11), 0.5 mg varenicline tablets as described herein, preferably as described in Example 6 and a second bottle comprising forty-two (42), 1.0 mg varenicline tablets as described herein, preferably as described in Example 6, and instructions for dosing/administration of the tablets. Preferably the instructions are printed and inform the user to: (i) begin treatment by orally administering one (1) 0.5 mg tablet once a day for the first three days; (ii) on days 4 to 7 of treatment, to orally administer one (1) 0.5 mg tablet twice a day (B.I.D.) or every twelve hours and (iii) on days 8 to 28 of treatment, to orally administer one (1) 1.0 mg tablet twice a day (B.I.D.) or every twelve hours wherein the eleven (11) 0.5 mg varenicline tablets are obtained from the first bottle in the kit and the forty-two (42), 1.0 mg varenicline tablets are obtained from the second bottle in the kit.

In further or alternative embodiments, the kit may further comprise a third and/or fourth bottle wherein the third and fourth bottles comprise fifty-six (56) 1.0 mg varenicline tablets as described herein, preferably as described in Example 6. The instructions may further inform the user to continue treatment after the eleven (11) 0.5 mg tablets in the first bottle and the forty-two (42) 1.0 mg tablets in the second bottle are consumed, by orally administering the 1.0 mg varenicline tablets twice a day (B.I.D.) or every twelve hours from day 29 to day 56 of treatment from the third bottle, if needed, and to further continue treatment by orally administering the 1.0 mg varenicline tablets twice a day (B.I.D.) or every twelve hours from day 57 to day 84 of treatment from the fourth bottle.

In another further embodiment, the kit may comprise three additional bottles, i.e., the fifth, sixth and seventh bottles, wherein each bottle comprises fifty-six (56) 1.0 mg varenicline tablets as described herein, preferably as described in Example 6 may be provided. The instructions may further inform the user to orally administering the 1.0 mg varenicline tablets twice a day (B.I.D.) or every twelve hours from day 85 to day 168 of treatment and to use all the tablets contained in the fifth bottle before opening the sixth bottle and to use all the tablets in the sixth bottle before opening the seventh bottle. In an alternative version, the three additional bottles, i.e., the fifth, sixth and seventh bottles may be packaged a separate or second kit with similar instructions for administration and opening of the individual bottles.

In yet another embodiment the kit may comprise: (i) a starter kit for use on days 1 to 28 of treatment which comprises a first bottle comprising eleven (11), 0.5 mg varenicline tablets as described herein, preferably as described in Example 6 and a second bottle comprising forty-two (42), 1.0 mg varenicline tablets as described herein, preferably as described in Example 6, and instructions for dosing/administration of the tablets in the starter kit as previously described; and (ii) optionally a maintenance kit comprising one, two, three, four or five bottles of 1.0 mg varenicline tablets as described herein, preferably as described in Example 6 and instructions informing the user to orally administering the 1.0 mg varenicline tablets twice a day (B.I.D.) or every twelve hours from day 29 to day 168 of treatment. The instructions should further inform the user to consume or use all the tablets contained in one of the bottles in the maintenance kit before opening a second or subsequent bottle that may be part of the maintenance kit. The starter kit and maintenance kits can be sold and/or distributed in a combined package, i.e. comprising both the starter and maintenance kits. Alternatively the starter kit and maintenance kits may be sold and/or distributed as separate kits.

In still further embodiments the kit may comprise: (i) a starter kit for use on days 1 to days 14, days 21, days 30 or longer such as days 31 to 70 and any day within the range of 30 to 70 such as day 37, 52, 57, 60, 65, 66, 67, 68, or 69 of treatment which comprises a first bottle comprising eleven (11), 0.5 mg varenicline tablets as described herein, preferably as described in Example 6 and a second bottle comprising a predetermined number of 1.0 mg varenicline tablets as described herein, preferably as described in Example 6, and instructions for dosing/administration of the tablets in the starter kit as previously described. The predetermined number of 1.0 mg varenicline tablets could be for example:
14, which will allow the twice a day administration of the 1.0 mg varenicline tablet from day 8 to day 14 of treatment;
28, which will allow the twice a day administration of the 1.0 mg varenicline tablet from day 8 to day 21 of treatment;
30, which will allow the twice a day administration of the 1.0 mg varenicline tablet from day 8 to day 22 of treatment;
46 which will allow the twice a day administration of the 1.0 mg varenicline tablet from day 8 to day 30 of treatment;
60 which will allow the twice a day administration of the 1.0 mg varenicline tablet from day 8 to day 37 of treatment;
90 which will allow the twice a day administration of the 1.0 mg varenicline tablet from day 8 to day 52 of treatment;
100 which will allow the twice a day administration of the 1.0 mg varenicline tablet from day 8 to day 57 of treatment;
106 which will allow the twice a day administration of the 1.0 mg varenicline tablet from day 8 to day 60 of treatment;
120 which will allow the twice a day administration of the 1.0 mg varenicline tablet from day 8 to day 67 of treatment; or any number of 1.0 mg varenicline tablets between 14 to 120.

The kit in this still further embodiment may also comprise a maintenance kit comprising one, two, three, four or five bottles of 1.0 mg varenicline tablets as described herein, preferably as described in Example 6 and instructions informing the user to orally administering the 1.0 mg varenicline tablets twice a day (B.I.D.) or every twelve hours from day 28 or whatever day the 1.0 mg varenicline tablets in the second bottle of the starter kit are consumed, e.g., day 14, day 21, day 30, day 37, day 52, day 57, day 60 or day 67 to day 168 of treatment. The instructions should further inform the user to consume or use all the tablets contained in one of the bottles in the maintenance kit before opening a second or subsequent bottle that may be part of the maintenance kit. The starter kit and maintenance kits can be sold and/or distributed in a combined package, i.e. comprising both the starter and maintenance kits. Alternatively the starter kit and maintenance kits may be sold and/or distributed as separate kits.

### DESCRIPTION OF THE PREFERRED EMBODIMENTS

The following are provided by way of example only and are by no means intended to be limiting.

### EXAMPLE 1

Stable varenicline tablets with the following composition were prepared:

| | | | | | |
|---|---|---|---|---|---|
| | | | mg/ | mg/ | |
| | | | tablet | tablet | % |
| Item | Ingredient | Function | (1 mg) | (0.5 mg) | Formula |

Tablet Core

| | | | | | |
|---|---|---|---|---|---|
| 1 | Varenicline | Active | 1.71 (eq. | 0.86 (eq. | 0.855% |
| | Tartrate | substance | to 1 mg | to 0.5 mg | |
| | | | Varen- | Varen- | |
| | | | icline) | icline) | |
| 2 | Microcrystalline | Filler/ | 120.00 | 60.00 | 60.0% |
| | Cellulose | Binder | | | |
| 3 | Anhydrous | Filler | 69.29 | 34.64 | 34.6% |
| | Dibasic | | | | |
| | Calcium | | | | |
| | Phosphate | | | | |
| 4 | Croscarmellose | Disintegrant | 6.00 | 3.00 | 3.0% |
| | Sodium | | | | |
| 5 | Colloidal Silicon | Glidant | 1.00 | 0.50 | 0.5% |
| | Dioxide | | | | |
| 6 | Magnesium | Lubricant | 2.00 | 1.00 | 1.0% |
| | Stearate | | | | |

| | | | |
|---|---|---|---|
| Sub-total | 200.00 | 100.00 | 100.0% |

Coating

| | | | | | |
|---|---|---|---|---|---|
| 7 | Tablet Core | Coating core | 200.00 | 100.00 | 95.2% |
| 8 | Opadry ® | Color-coating | 6.00 | n/a | 2.9% |
| | Blue | polymer | | | |
| 9 | Opadry ® | Color-coating | n/a | 3.00 | |
| | White | polymer | | | |
| 10 | Opadry ® | Coating | 4.00 | 2.00 | 1.9% |
| | | polymer/ | | | |
| | Clear | isolation layer | | | |

| | | | |
|---|---|---|---|
| Total | 210.00 | 105.00 | 100.0% |

### Formula of Final Blend (Common Blend for 0.5 mg and 1 mg)

| | Theoretical Amount |
|---|---|
| Raw Material Name | Required (kg) |
| Varenicline Tartrate | 0.7268 |
| Microcrystalline Cellulose, NF | 51.00 |
| Anhydrous Dibasic Calcium | 29.45 |
| Phosphate, USP | |
| Croscarmellose Sodium, NF | 2.550 |
| Colloidal silicon Dioxide, USP/NF | 0.4250 |
| Magnesium Stearate, USP/NF | 0.8500 |
| Batch size of the final blend | 85.00 |

### Coating Formula for 0.5 mg

| | | Theoretical Amount |
|---|---|---|
| Material Name | | Required (kg) |
| Varenicline Tablets (0.5 mg) | | 42.50 |
| First | Clear Opadry ® Clear | 0.850 |
| Layer | Purified Water, USP | 7.65 |
| Second | White Opadry ® White | 1.275 |
| Layer | Purified Water, USP | 7.23 |

| | |
|---|---|
| Total Amount of Coated Tablets | 44.625 |

### Coating Formula for 1 mg

| | | Theoretical Amount |
|---|---|---|
| Material Name | | Required (kg) |
| Varenicline Tablets (1 mg) | | 42.50 |
| First | Clear Opadry ® Clear | 0.850 |
| Layer | Purified Water, USP | 7.65 |
| Second | White Opadry ® Blue | 1.275 |
| Layer | Purified Water, USP | 7.23 |

| | |
|---|---|
| Total Amount of Coated Tablets | 44.625 |

The water content of anhydrous Dibasic Calcium Phosphate is typically 0.1-0.2%.

The Opadry® Blue was Opadry® 03B20966 and contained hydroxypropyl methylcellulose, titanium dioxide, polyethylene glycol, talc, FD&C blue #2/indigo Carmine Aluminum Lake.

The Opadry® White was Opadry® 03B28796 and contained hydroxypropyl methylcellulose, titanium dioxide and polyethylene glycol.

The Opadry® Clear was Opadry® 03K19229 and contained hydroxypropyl methylcellulose, triacetin and talc.

All the Opadry coatings were obtained from Shanghai Colorcon Coating Technology Limited, China.

The stable tablets were prepared using the following method:

(i) varenicline and anhydrous dibasic calcium phosphate (approximately two times the amount of the varenicline) are added to a plastic bag and mixed in a plastic bag. The varenicline and anhydrous dibasic calcium phosphate mixture is screened through a #30 mesh screen and added to a bin blender containing about one half the amount of microcrystalline cellulose and blended.

(ii) the croscarmellose sodium, remaining amount of microcrystalline cellulose and the remaining amount of anhydrous dibasic calcium phosphate are added to the mixture prepared in step (i) and blended to create a pre-blend.

(iii) the pre-blend of step (ii) is dry granulated using a roller compactor.

(iv) the dry granules of step (iii) are added to the bin blender along with colloidal silicone dioxide and magnesium stearate that has been screened through a #30 mesh screen and blended to create a final blend.

(v) the final blend of step (iv) is compressed into core tablets, containing 0.5 mg varenicline free base or 1.0 mg of varenicline free base.

(vi) the core tablets are first coated with an Opadry® clear seal coating then an Opadry® color coating using a pan coater.

The particle size of five representative lots of varenicline tartrate was determined and the results are as follows:

| Lot No. | D10 (μm) | D50 (μm) | D90 (μm) |
|---|---|---|---|
| 202012001 | 1.547 | 4.088 | 8.554 |
| 202012002 | 2.269 | 5.708 | 11.288 |
| 202010004 | 2.339 | 6.354 | 11.802 |
| 201810001 | 0.858 | 2.600 | 9.660 |
| 201811001 | 0.878 | 2.630 | 32.700 |

The tablets are packaged in 40 cc HDPE bottles with a 33 mm neck, sealed and closed with a 33 mm child resistant cap. The tablets may also be packaged in a blister package comprising a PCTFE/PVC composite blister sheet with an aluminium foil backing.

The 0.5 mg tablets will have a total nitrosamine impurity content of less than 18.5 ppm, less than 15.5 ppm, preferably less than 15 ppm, 14 ppm, 13 ppm, 12 ppm, 11 ppm, 10 ppm, 9 ppm, 8 ppm, 7 ppm, 6 ppm or 5 ppm when manufactured and after storage in the HDPE bottles or blister packs for at least 6 months or longer at approximately 25° C. and approximately 60% relative humidity or for at least three months or longer at approximately 40° C. and approximately 75% relative humidity.

The 1 mg tablets will have a total nitrosamine impurity content of less than 18.5 ppm, less than 15.5 ppm, preferably less than 15 ppm, 14 ppm, 13 ppm, 12 ppm, 11 ppm, 10 ppm, 9 ppm, 8 ppm, 7 ppm, 6 ppm or 5 ppm when manufactured and after storage in the HDPE bottles or blister packs for at least 6 months or longer at approximately 25° C. and approximately 60% relative humidity or for at least three months or longer at approximately 40° C. and approximately 75% relative humidity.

The varenicline tartrate employed in the above had a particle size, D90 ranging from about 2 μm to about 35 μm, preferably about 2 μm to about 20 μm and a D50 ranging from about 2 μm to about 10 μm.

### EXAMPLE 2

Three lots of the 0.5 mg and 1 mg tablets described in Example 1 were prepared. Samples of each lot were packaged in a 40 cc HDPE bottle that was sealed and capped with a lined child resistant cap. Samples of each lot were also packaged in PCTFE/PVC composite blisters and sealed with aluminum foil. The moisture content of the samples were measured and determined using the method described in the United States Pharmacopeia (USP) section <921>. The moisture content is shown in the following tables after storage at 25° C. and 60% relative humidity:

| | 0.5 mg, blister | | | 0.5 mg, bottle | | |
|---|---|---|---|---|---|---|
| LOT # | 181104-1A | 181105-1A | 181106-1A | 181104-1B | 181105-1B | 181106-1B |
| T = 0 M | 2.5% | 2.3% | 2.8% | 2.5% | 2.3% | 2.8% |
| T = 3 M | 3.4% | 3.7% | 3.5% | 4.1% | 4.0% | 3.4% |
| T = 6 M | 3.0% | 3.3% | 3.6% | 3.0% | 2.8% | 3.4% |

| | 1 mg, blister | | | 1 mg, bottle | | |
|---|---|---|---|---|---|---|
| LOT # | 181104-2A | 181105-2A | 181106-2A | 181104-2B | 181105-2B | 181106-2B |
| T = 0 M | 2.8% | 2.8% | 2.8% | 2.8% | 2.8% | 2.8% |
| T = 3 M | 3.8% | 4.2% | 4.3% | 3.8% | 3.9% | 3.9% |
| T = 6 M | 3.1% | 3.5% | 2.4% | 2.9% | 3.0% | 3.1% |

The impurity levels were also determined and found to be as follows:

### Total Impurity Data (Nitrosamines Not Included)

| | | | | | | | | |
|---|---|---|---|---|---|---|---|---|
| Lot No. | Condition | T = 0 M | T = 3 M | T = 6 M | T = 9 M | T = 12 M | T = 18 M | T = 24 M |

0.5 mg blister

| | | | | | | | | |
|---|---|---|---|---|---|---|---|---|
| 181104-1A | 25° C./60% RH | ND | <LOQ | 0.1% | 0.06% | 0.1% | 0.2% | 0.3% |
| | 40° C./75% RH | ND | 0.2% | 0.5% | | | | |
| 181105-1A | 25° C./60% RH | <LOQ | <LOQ | <LOQ | 0.07% | 0.1% | 0.2% | 0.3% |
| | 40° C./75% RH | <LOQ | 0.2% | 0.8% | | | | |
| 181106-1A | 25° C./60% RH | 0.1% | 0.05% | 0.3% | 0.06% | 0.1% | 0.2% | 0.2% |
| | 40° C./75% RH | 0.1% | 0.2% | 0.5% | | | | |

0.5 mg bottle

| | | | | | | | | |
|---|---|---|---|---|---|---|---|---|
| 181104-1B | 25° C./60% RH | ND | <LOQ | 0.1% | <LOQ | <LOQ | 0.1% | 0.2% |
| | 40° C./75% RH | ND | 0.1% | 0.2% | | | | |
| 181105-1B | 25° C./60% RH | <LOQ | 0.1% | 0.1% | <LOQ | <LOQ | 0.1% | 0.2% |
| | 40° C./75% RH | <LOQ | 0.1% | 0.3% | | | | |
| 181106-1B | 25° C./60% RH | 0.1% | <LOQ | <LOQ | 0.05% | 0.05% | 0.1% | 0.2% |
| | 40° C./75% RH | 0.1% | 0.1% | 0.3% | | | | |

1 mg blister

| | | | | | | | | |
|---|---|---|---|---|---|---|---|---|
| 181104-2A | 25° C./60% RH | <LOQ | <LOQ | <LOQ | 0.1% | 0.05% | 0.1% | 0.1% |
| | 40° C./75% RH | <LOQ | 0.1% | 0.3% | 0.05% | 0.05% | 0.2% | 0.5% |
| 181105-2A | 25° C./60% RH | <LOQ | <LOQ | <LOQ | | | | |
| | 40° C./75% RH | <LOQ | 0.1% | 0.3% | | | | |
| 181106-2A | 25° C./60% RH | <LOQ | <LOQ | 0.05% | <LOQ | 0.1% | 0.1% | 0.2% |
| | 40° C./75% RH | <LOQ | 0.1% | 0.4% | | | | |

1 mg bottle

| | | | | | | | | |
|---|---|---|---|---|---|---|---|---|
| 181104-2B | 25° C./60% RH | <LOQ | <LOQ | <LOQ | <LOQ | <LOQ | <LOQ | <LOQ |
| | 40° C./75% RH | <LOQ | 0.1% | 0.2% | | | | |
| 181105-2B | 25° C./60% RH | <LOQ | <LOQ | 0.1% | <LOQ | <LOQ | <LOQ | 0.2% |
| | 40° C./75% RH | <LOQ | 0.05% | 0.2% | | | | |
| 181106-2B | 25° C./60% RH | <LOQ | <LOQ | 0.1% | <LOQ | <LOQ | <LOQ | 0.06% |
| | 40° C./75% RH | <LOQ | 0.1% | 0.2% | | | | |

### Varenicline Tartrate Bulk Drug Substance (API)

| API Lot No | Storage condition/ Time | 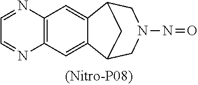 | 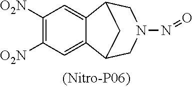 [SMILES: O=NN1CC2CC(C1)c1cc([N+](=O)[O-])c([N+](=O)[O-])cc12] | 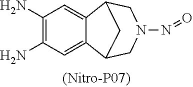 [SMILES: Nc1cc2c(cc1N)C1CC2CN(N=O)C1] |
|---|---|---|---|---|
| Lot | initial | 1.6 ppm | <LOQ (0.0289 ppm) | ND* |
| 202101002 | | | | |
| Lot | <30° C., | 7.1 ppm | <LOQ (0.0289 ppm) | ND* |
| 201810001 | >30 M | | | |
| Lot | <30° C., | 18.3 ppm | <LOQ (0.0289 ppm) | ND* |
| 201811001 | >30 M | | | |

*LOD 0.1344 ppm, LOQ 0.2688 ppm.

### Varenicline Tablets (1 mg in Blister)

| API Lot No | Finish product lot No. | Storage condition/Time | 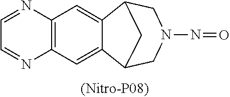 [SMILES: O=NN1CC2CC(C1)c1cc3nccnc3cc12] |
|---|---|---|---|
| Lot 201810001 | 181104-2A | 25° C./60% RH/24 M** | 23 ppm |
| Lot 201810001 | 181105-2A | 25° C./60% RH/24 M** | 28 ppm |
| Lot 201811001 | 181106-2A | 25° C./60% RH/24 M** | 63 ppm |

**25° C./60RH/24 M + room temperature about 9 months.

### Varenicline Tablets (1 mg in Bottle)

| API Lot No | Finish product lot No. | Storage condition/Time | 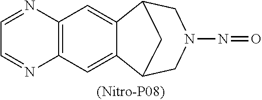 [SMILES: O=NN1CC2CC(C1)c1cc3nccnc3cc12] |
|---|---|---|---|
| Lot 201810001 | 181104-2B | 25° C./60% RH/24 M** | 22 ppm |
| Lot 201810001 | 181105-2B | 25° C./60% RH/24 M** | 34 ppm |
| Lot 201811001 | 181106-2B | 25° C./60% RH/24 M** | 72 ppm |

**25° C./60RH/24 M + room temperature about 9 months.

### EXAMPLE 3

A varenicline excipient compatibility study was conducted using the varenicline tartrate and Opadry® coatings described below. The varenicline tartrate and Opadry® were hand mixed in a sealed bottle for about two minutes and the following results were obtained:

| | API: | Total impurity | | |
|---|---|---|---|---|
| Coating | Coating | (Nitrosamines not included) | | |
| Materials | Materials | initial | 7 days | 14 days |
| Opadry ® Blue | 1:5 | ND | 0.06 | 0.18 |
| Opadry ® White | 1:5 | ND | 0.17 | 0.23 |
| Opadry ® Clear | 1:5 | ND | ND | ND |

| API LOT No. | D10 | D50 | D90 |
|---|---|---|---|
| 201702001 (80 mesh) | 1.1 | 3.3 | 11.0 |

### EXAMPLE 4

A varenicline manufacturing study was conducted to evaluate the mixing order on nitrosamine levels. In the study, the core excipients described in Example 1, without the lubricant, were, mixed with the varenicline, with and without the pre-blending step. The nitrosamine impurities were measured after the blends were stored in a sealed HDPE bottle at 60° C. and 75% relative humidity:

| | | Nitrosamines | |
|---|---|---|---|
| | | impurity | |
| | | (Nitro-P08) | |
| | | | 60° C./ |
| | | Initial | 75% RH |
| LOT | Pre-blending Process | API | 14 days |
| VL2106301C1 | De-lumping anhydrous dibasic | 1.4 | 5.0 |
| | calcium phosphate with API first, | ppm | ppm |
| | then pre-blending with other | | |
| | excipients except lubricant. | | |
| VL2106302C1 | Pre-blending excipients except | | 8.9 |
| | lubricant with API. | | ppm |

The “initial” value refers to the value of the bulk varenicline tartrate without excipients that was used to prepare the described test compositions. The initial value was determined at the same time as the described test sample, however, the bulk varenicline tartrate had been stored in a sealed container at about 2-8° C. The total process time for VL2106301C1 was about 31 hours and about 52 hours for VL2106302C1.

The data in the above table demonstrates that pre-blending of varenicline with a low initial total nitrosamine level with a low moisture excipient slows the rate at which the nitrosamine levels increase.

### EXAMPLE 5

A varenicline manufacturing study was conducted to evaluate the composition of the first coating layer, i.e. the coating in contact with the tablet core. Varenicline tartrate tablet cores were prepared as described in example 1. VL2106301C2 coated Opadry® Blue only and the coating time was 30 min shorter than VL2106301C1. The nitrosamine impurities is shown in the following tables with or without the coating comprises a low viscosity polyvinyl alcohol or hydroxpropyl methylcellulose (aka hypromellose) without a pigment or polyethylene glycol and storage at 60° C. and 75% relative humidity:

| | | Nitrosamines impurity | |
|---|---|---|---|
| | | (Nitro-P08) in tablets | |
| | | | 14 days, |
| LOT | Coating | initial | bottling |
| VL2106301 | Tablet core without coating | 3.2 ppm | 23.9 ppm |
| VL2106301 | 1^{st} coating: clear coat | 1.4 ppm | 5.0 ppm |
| C1 | 2^{nd} coating: color coat | | |
| VL2106301 | 1^{st} coating: color coat | N/A | 5.0 ppm |
| C2 | 2^{nd} coating: N/A | | |

The “initial” value refers to the value of the bulk varenicline tablets (without packaged with induction seal). The initial value was determined at the same time as the described test sample, however, the bulk varenicline tablets had been stored in a sealed container at about 2-8° C. The total process time for VL2106301 was about 3 hours and about 31 hours for VL2106301C1 and about 33.5 hours for VL2106301C2.

### EXAMPLE 6

A study was conducted to evaluate nitrosamine levels during manufacturing using two different lots of varenicline tartrate with different initial total nitrosamine levels. The two lots and initial total nitrosamine levels were as follows:

| Example No. | API nitrosamine level (Nitro-P08) |
|---|---|
| (API Lot No.) | (based on varenicline base) (ppm) |
| 6A | 7.35 |
| (202001001R1) | |
| 6B | 1.8 |
| (202012002) | |

The varenicline tartrate of Example 6A and 6B was used to prepare the intermediate API/excipient blend (including lubricant), the intermediate core tablet and intermediate coated tablet core with the following composition:

| Example 6A | | Example 6B | | | |
|---|---|---|---|---|---|
| 1 mg | | 1 mg | | 0.5 mg | |
| mg/tablet | wt % | mg/tablet | wt % | mg/tablet | wt % |

Blend

| | | | | | | |
|---|---|---|---|---|---|---|
| Varenicline tartrate | 1.71 | 0.8 | 1.71 | 0.8 | 0.855 | 0.8 |
| Anhydrous Dibasic | 120.00 | 57.1 | 120.00 | 57.1 | 60.00 | 57.1 |
| Calcium Phosphate | | | | | | |
| Microcrystalline Cellulose | 69.29 | 33.0 | 69.29 | 33.0 | 34.645 | 33.0 |
| Croscarmellose Sodium | 6.00 | 2.9 | 6.00 | 2.9 | 3.00 | 2.9 |
| Colloidal Silicon Dioxide | 1.00 | 0.5 | 1.00 | 0.5 | 0.50 | 0.5 |
| Magnesium Stearate | 2.00 | 1.0 | 2.00 | 1.0 | 1.00 | 1.0 |

First Coating

| | | | | | | |
|---|---|---|---|---|---|---|
| Opadry ® Clear | 4.00 | 1.9 | 4.00 | 1.9 | 2.00 | 1.9 |

Second Coating

| | | | | | | |
|---|---|---|---|---|---|---|
| Opadry ® Blue | 6.00 | 2.9 | 6.00 | 2.9 | N/A | N/A |
| Opadry ® White | N/A | N/A | N/A | N/A | 3.00 | 2.9 |
| Total | 210.0 | 100.0 | 210.0 | 100.0 | 105.0 | 100.0 |

The intermediate blend, intermediate core tablet and intermediate coated tablet were prepared as described in Examples 1 and 2. 200 mg of the Example 6A intermediate blend, 200 mg of the Example 6A (1 mg) intermediate core tablets and 210 mg of the Example 6A (1 mg) coated tablet were placed in a 400 L s/s transfer bins double-lined with clean LDPE bags, place 2 desiccant bags (125g each) between two PE bags, and closed the bags and the lid securely and stored under ambient conditions, i.e. room temperature, for 30 days. The total nitrosamine and moisture content of the samples over time was determined and reported below:

| | Example 6A - Intermediate Products | | | | | |
|---|---|---|---|---|---|---|
| ambient | | Core | Coated | | | |
| conditions | Blend | Tablets | Tablets | | Core | Coated |
| No. of | N-Nitroso Varenicline | | | Blend | Tablets | Tablets |
| Days | (Nitro-P08) (unit: ppm) | | | Moisture Content (%) | | |
| 0 | 9.1 | 9.0 | 11.9 | 2.3 | 2.6 | 2.2 |
| 7 | 11.1 | 12.9 | 10.6 | 2.5 | 2.7 | 2.5 |
| 14 | 14.0 | 15.2 | 11.4 | 2.5 | 2.5 | 1.7 |
| 30 | 15.4 | 15.4 | 14.4 | 2.0 | N/A | N/A |

200 mg of the Example 6B intermediate blend, 200 mg of the 1 mg Example 6B intermediate core tablet and 210 mg of the 1 mg Example 6B coated tablet were placed in a 400 L s/s transfer bins double-lined with clean LDPE bags, placed 2 desiccant bags (125g each) between two PE bags, and closed the bags and the lid securely and stored under ambient conditions, i.e. room temperature, for 60 days. The total nitrosamine content of the samples over time was determined and reported below:

| | Example 6B - Intermediate Products | | | | | |
|---|---|---|---|---|---|---|
| ambient | | Core | Coated | | | |
| conditions | Blend | Tablets | Tablets | | Core | Coated |
| No. of | N-Nitroso Varenicline | | | Blend | Tablets | Tablets |
| Days | (Nitro-P08) (unit: ppm) | | | Moisture Content (%) | | |
| 0 | 3.9 | 4.9 | 4.9 | 3.0 | 3.2 | 2.4 |
| 7 | 4.3 | 6.0 | 5.4 | 2.9 | 3.1 | 2.6 |
| 14 | 4.1 | 6.3 | 5.0 | 2.5 | 2.6 | 2.4 |
| 30 | 7.3 | 8.7 | 6.6 | 2.7 | 3.0 | 2.4 |
| 90 | 4.3 | 6.3 | 6.7 | 2.6 | 2.6 | 2.1 |

### Bottle Configuration

56 tablets of the 1 mg and 0.5 mg coated tablets of Example 6B were placed in a 40 cc HDPE bottles with and without desiccants and sealed. The sealed bottles with varying amounts of desiccant were stored at 60° C. and 75% relative humidity and the total nitrosamine and moisture content was determined and reported below:

### 1 mg Final Tablet—Example 6B

| Stability | Amount of Silica Gel | | | | | |
|---|---|---|---|---|---|---|
| Condition: | (canister) | | | Amount of Silica Gel | | |
| 60° C./ | Desiccant Per Bottle | | | (canister) | | |
| 75% RH | 0 g | 2 g | 4 g | Desiccant Per Bottle | | |
| No. of | N-Nitroso Varenicline | | | 0 g | 2 g | 4 g |
| Days | (Nitro-P08) (unit: ppm) | | | Moisture Content (%) | | |
| Initial | 4.9 | | | 3.2 | | |
| 7 | 20.1 | 10.0 | 8.6 | 2.3 | 2.0 | 2.0 |
| 14 | 22.3 | 13.2 | 10.3 | 2.3 | 2.0 | 1.9 |
| 21 | 28.7 | 13.1 | 11.1 | 2.6 | 2.1 | 1.9 |

### 0.5 mg Final Tablet—Example 6B

| Stability | Amount of Silica Gel | | | | | |
|---|---|---|---|---|---|---|
| Condition: | (canister) | | | Amount of Silica Gel | | |
| 60° C./ | Desiccant Per Bottle | | | (canister) | | |
| 75% RH | 0 g | 2 g | 4 g | Desiccant Per Bottle | | |
| No. of | N-Nitroso Varenicline | | | 0 g | 2 g | 4 g |
| Days | (Nitro-P08) (unit: ppm) | | | Moisture Content (%) | | |
| Initial | 5.5 | | | 2.5 | | |
| 7 | 26.1 | 11.4 | 10.5 | 2.9 | 2.6 | 2.7 |
| 14 | 30.7 | 13.4 | 12.2 | 2.8 | 2.2 | 2.0 |
| 21 | 28.3 | 12.5 | 11.3 | 2.4 | 1.9 | 1.8 |

56 tablets of the 1 mg coated tablets of Example 6A were placed in a 40 cc HDPE bottles with and without desiccants and sealed. The sealed bottles with varying amounts of desiccant were stored at 60° C. and 75% relative humidity and the total nitrosamine and moisture content was determined and reported below:

### 1 mg Final Tablet—Example 6A

| Stability | | | | | | | | |
|---|---|---|---|---|---|---|---|---|
| Condition: | Number of Silica Gel (bag) | | | | | | | |
| 60° C./ | Desiccant Per Bottle | | | | Number of Silica Gel (bag) | | | |
| 75% RH | 0 g | 1 g | 2 g | 4 g | Desiccants Per Bottle | | | |
| No. of | N-Nitroso Varenicline | | | | 0 g | 1 g | 2 g | 4 g |
| Days | (Nitro-P08) (unit: ppm) | | | | Moisture Content (%) | | | |
| Initial | 11.9 | | | | 2.2 | | | |
| 7 | 34.4 | 20.4 | 15.2 | 20.0 | 2.0 | 1.7 | 1.5 | 1.7 |
| 14 | 34.8 | 24.4 | 17.3 | 17.1 | 2.1 | 1.8 | 2.2 | 2.8 |

56 and 42 tablets of the 1 mg coated tablets and 56 and 11 tablets of the 0.5 mg coated tablets of Example 6B were placed in a 40 cc HDPE bottles with and without desiccants and sealed. The sealed bottles with varying amounts of desiccant and tablets were stored at 40° C. and 75% relative humidity and the total nitrosamine and moisture content was determined and reported below:

### 1 mg Final Tablet (56-Count)—Example 6B

| | | Amount of Silica Gel | | | | | |
|---|---|---|---|---|---|---|---|
| Stability | | (canister) | | | Amount of Silica Gel | | |
| Condition: | | Desiccant Per Bottle | | | (canister) | | |
| 40° C./ | | 56-count | | | Desiccant Per Bottle | | |
| 75% RH | | 0 g | 2 g | 4 g | 56-count | | |
| No. of | | N-Nitroso Varenicline | | | 0 g | 2 g | 4 g |
| Months | | (Nitro-P08) (unit: ppm) | | | Moisture Content (%) | | |
| Initial | | 6.5 | 5.7 | 5.9 | 2.8 | 2.4 | 2.7 |
| 0.5 | M | 12.8 | 7.3 | 7.2 | 2.4 | 2.0 | 1.9 |
| 1.5 | M | 19.2 | 11.3 | 9.4 | 2.2 | 1.9 | 1.6 |
| 2 | M | 16.9 | 10.4 | 9.5 | 2.4 | 2.1 | 2.0 |
| 2.5 | M | 19.4 | 9.7 | 10.0 | 2.8 | 2.6 | 2.2 |
| 3 | M | N/A | 11.4 | 10.3 | N/A | 2.1 | 1.8 |
| 4 | M | N/A | 12.4 | 11.1 | N/A | 2.0 | 2.2 |

1 mg Final Tablet (42-Count)—Example 6B

| | | Amount of Silica Gel | | | | | |
|---|---|---|---|---|---|---|---|
| Stability | | (canister) | | | Amount of Silica Gel | | |
| Condition: | | Desiccant Per Bottle | | | (canister) | | |
| 40° C./ | | 42-count | | | Desiccant Per Bottle | | |
| 75% RH | | 0 g | 2 g | 4 g | 42-count | | |
| No. of | | N-Nitroso Varenicline | | | 0 g | 2 g | 4 g |
| Months | | (Nitro-P08) (unit: ppm) | | | Moisture Content (%) | | |
| Initial | | 6.8 | 6.7 | 6.1 | 2.8 | 2.5 | 2.3 |
| 0.5 | M | 12.6 | 7.2 | 6.4 | 2.5 | 3.0 | 2.7 |
| 1.5 | M | 17.9 | 10.9 | 9.9 | 2.2 | 2.9 | 1.4 |
| 2 | M | 17.0 | 9.9 | 9.1 | 2.7 | 2.3 | 2.2 |
| 2.5 | M | 18.4 | 9.1 | 7.9 | 2.7 | 2.3 | 2.1 |
| 3 | M | N/A | 9.7 | 10.6 | N/A | 2.1 | 2.3 |
| 4 | M | N/A | 13.0 | 11.3 | N/A | 2.5 | 1.9 |

### 0.5 mg Final Tablet (56-Count)—Example 6B

| | | Amount of Silica Gel | | | | | |
|---|---|---|---|---|---|---|---|
| Stability | | (canister) | | | Amount of Silica Gel | | |
| Condition: | | Desiccant Per Bottle | | | (canister) | | |
| 40° C./ | | 56-count | | | Desiccant Per Bottle | | |
| 75% RH | | 0 g | 2 g | 4 g | 56-count | | |
| No. of | | N-Nitroso Varenicline | | | 0 g | 2 g | 4 g |
| Months | | (Nitro-P08) (unit: ppm) | | | Moisture Content (%) | | |
| Initial | | 6.7 | 5.7 | 5.8 | 2.2 | 2.1 | 1.7 |
| 0.5 | M | 11.7 | 7.6 | 7.6 | 2.2 | 2.5 | 2.9 |
| 1.5 | M | 20.9 | 10.3 | 9.9 | 2.0 | 1.4 | 1.5 |
| 2 | M | 20.9 | 11.1 | 10.3 | 2.0 | 1.6 | 1.5 |
| 2.5 | M | 23.5 | 11.6 | 10.4 | 2.6 | 2.4 | 2.1 |
| 3 | M | N/A | 11.1 | 10.0 | N/A | 2.0 | 2.0 |
| 4 | M | N/A | 11.9 | 11.3 | N/A | 2.5 | 2.6 |

### 0.5 mg Final Tablet (11-Count)—Example 6B

| | | Amount of Silica Gel | | | | | |
|---|---|---|---|---|---|---|---|
| Stability | | (canister) | | | Amount of Silica Gel | | |
| Condition: | | Desiccant Per Bottle | | | (canister) | | |
| 40° C./ | | 11-count | | | Desiccant Per Bottle | | |
| 75% RH | | 0 g | 2 g | 4 g | 11-count | | |
| No. of | | N-Nitroso Varenicline | | | 0 g | 2 g | 1 g |
| Months | | (Nitro-P08) (unit: ppm) | | | Moisture Content (%) | | |
| Initial | | 6.7 | 5.2 | 5.4 | 2.5 | 2.6 | 1.6 |
| 0.5 | M | 13.5 | 7.1 | 7.7 | 2.5 | 2.0 | 1.5 |
| 1.5 | M | 29.2 | 8.8 | 8.3 | 2.6 | 1.3 | 1.2 |
| 2 | M | 29.3 | 9.9 | 8.9 | 3.0 | 1.3 | 1.2 |
| 2.5 | M | 27.8 | 8.7 | 7.5 | 3.3 | 2.8 | 2.6 |
| 3 | M | N/A | 8.8 | 8.4 | N/A | 2.0 | 1.8 |
| 4 | M | N/A | 11.5 | 9.9 | N/A | 2.2 | 2.2 |

The absorption rate of desiccant employed in above bottle configurations is shown in the table below.

| | | RH 20% | RH 40% | RH 50% | RH 80% | RH 90% |
|---|---|---|---|---|---|---|
| Absorption | Canister | — | 19% | — | 27% | — |
| Rate (%) | Desiccants | | | | | |
| (under 25° C.) | Silica Gel | 7% | — | 19% | — | 29% |
| | Desiccants bag | | | | | |

The foregoing absorption rate information may be used to calculate the absorption capacity according to the equation:

Absorption Capacity (g)=Desiccant Amount (g)×Absorption Rate (%)

Using the above data and specifically the 2 g desiccant in 40 cc HDPE bottle as an example, it is believed that desiccants in an amount to exhibit an Absorption Capacity (under 25° C.) of:
0.035 g per 10 ml volume of container under RH20%;
0.095 g per 10 ml volume of container under RH40%;
0.095 g per 10 ml volume of container under RH50%;
0.135 g per 10 ml volume of container under RH80%; and/or
0.145 g per 10 ml volume of container under RH90%

should produce acceptable stability.

### Blister Configuration

The 1 mg coated tablets of Example 6B were packaged in PVC or PCTFE/PVC composite sheet blister that were sealed with aluminum foil to form a 7 count blister card. 4 of the 7 count blister cards were placed in an aluminum foil bag [Structure (thickness): PET (12 μm)/AL (7 μm)/CPP (70 μm)] along with 16 grams of silica gel desiccant bags and sealed. The sealed bags with the blister card and desiccant were stored at 60° C. and 75% relative humidity and the total nitrosamine and moisture content was determined and reported below:

| | Blister Forming Material | | | |
|---|---|---|---|---|
| | Stability Condition: 60° C./75% RH | | | |
| | PVC | PCTFE/PVC | PVC | PCTFE/PVC |
| | sheet | composite sheet | sheet | composite sheet |
| | N-Nitroso Varenicline | | Moisture | |
| No. of Days | (Nitro-P08) (unit: ppm) | | Content (%) | |
| Initial | 4.9 | | 2.4 | |
| 7 | 14.5 | 20.8 | 1.7 | 2.0 |
| 14 | 14.7 | 23.7 | 1.4 | 1.6 |
| 21 | 17.1 | 28.2 | 1.4 | 1.2 |

The PCTFE sheet is commercially available under the tradename ACLAR® 2000 from Honeywell Life Science division. The PCTFE can be combined with PVC to form the PCTFE/PVC composite sheet. The properties of the PVC and PCTFE/PVC composite sheet are as follows:

| | | Water Vapor |
|---|---|---|
| | | Transmission |
| Blister Forming Material | Thickness (μm) | Rate [g/m² * d] |
| PCTFE (ACLAR ® | 305 (PCTFE: 51 μm, | ≤0.11 |
| 2000)/PVC sheet | PVC: 250 μm) | |
| PVC sheet | 250 | ≤2.5 |

Based on the above date, it is believed that blister forming material with a thickness of 250 μm or below should produce acceptable stability and it is believed that blister forming material exhibits a water vapor transmission rate (WVTR) at least 0.11 g/m²*day, preferably exhibits a WVTR at least 0.25 g/m²*day should produce acceptable stability.

The 1 mg coated tablets of Example 6B were packaged in PVC or PCTFE/PVC composite sheet blister that were sealed with aluminum foil to form a 28 count blister card. 2 of the 28 count blister cards were placed in an aluminum foil bag with or without canister desiccants and sealed. The aluminum foil bag with the blister card and desiccant were stored at 60° C. and 75% relative humidity and 40° C. and 75% relative humidity, respectively. The total nitrosamine and moisture content was determined and reported below:

| | Blister Forming Material | | | | | | | |
|---|---|---|---|---|---|---|---|---|
| | PCTFE/ | PCTFE/ | | | | | | |
| | PVC | PVC | PVC | PVC | PCTFE/ | PCTFE/ | | |
| Stability | Without | With 6 g | With 6 g | With 12 g | PVC | PVC | PVC | PVC |
| Condition: | Dessicant | Canister | Canister | Canister | Without | With 6 g | With 6 g | With 12 g |
| 60° C./ | Without Al | Two blisters and dessicants were | | | Dessicant | Canister | Canister | Canister |
| 75% RH | foil bag | sealed into an Al foil bag | | | Without Al | Two blisters and dessicants were | | |
| No. of | N-Nitroso Varenicline | | | | foil bag | sealed into an Al foil bag | | |
| Days | (Nitro-P08) (unit: ppm) | | | | Moisture Content (%) | | | |
| Initial | 4.8 | | | | 2.6 | | | |
| 7 | 32.5 | 24.6 | 24.3 | 23.3 | 2.5 | 1.9 | 1.3 | 1.6 |
| 14 | 40.9 | 29.4 | 27.7 | 24.8 | 2.5 | 1.7 | 1.3 | 1.2 |
| 21 | 44.6 | 32.0 | 25.7 | 24.3 | 2.8 | 2.0 | 1.3 | 1.3 |

| | | Blister Forming Material | | | | | | | |
|---|---|---|---|---|---|---|---|---|---|
| | | PCTFE/ | PCTFE/ | | | | | | |
| | | PVC | PVC | PVC | PVC | PCTFE/ | PCTFE/ | | |
| Stability | | Without | With 6 g | With 6 g | With 12 g | PVC | PVC | PVC | PVC |
| Condition: | | Dessicant | Canister | Canister | Canister | Without | With 6 g | With 6 g | With 12 g |
| 40° C./ | | Without Al | Two blisters and dessicants were | | | Dessicant | Canister | Canister | Canister |
| 75% RH | | foil bag | sealed into an Al foil bag | | | Without Al | Two blisters and dessicants were | | |
| No. of | | N-Nitroso Varenicline | | | | foil bag | sealed into an Al foil bag | | |
| Months | | (Nitro-P08) (unit: ppm) | | | | Moisture Content (%) | | | |
| Initial | | 4.8 | | | | 2.6 | | | |
| 0.5 | M | 16.9 | 14.9 | 15.3 | 13.9 | 2.2 | 2.0 | 1.7 | 1.7 |
| 1 | M | 12.4 | 17.6 | 17.3 | 16.6 | 2.3 | 1.9 | 1.4 | 1.3 |
| 1.5 | M | 21.6 | 15.1 | 14.3 | 15.5 | 2.8 | 2.0 | 2.0 | 1.9 |
| 2 | M | 19.5 | 14.9 | 14.5 | 13.8 | N/A | 1.8 | 1.4 | 1.4 |
| 2.5 | M | N/A | 16.3 | 16.3 | 15.9 | N/A | 2.2 | 1.6 | 2.6 |
| 3 | M | N/A | 17.0 | 16.3 | 17.3 | N/A | 2.2 | 2.1 | 2.0 |

The absorption rate of desiccant employed in above blister configurations is shown in the table below.

| | | RH 20% | RH 40% | RH 50% | RH 80% | RH 90% |
|---|---|---|---|---|---|---|
| Absorption Rate (%) | Canister | — | 19% | — | 27% | — |
| (under 25° C.) | Desiccants | | | | | |

The foregoing absorption rate information may be used to calculate the absorption capacity according to the equation:

Absorption Capacity (g)=Desiccant Amount (g)×Absorption Rate (%)

Using the above date and specifically the 6 g Desiccants and two blisters (each with a dimension of 8.5 cm×7.35 cm×0.465 cm=29 cm³) together in an Aluminum bag (container volume is assumed to be about 60 cm³, i.e. slightly larger than the volume of the two blister cards) as an example, it is believed that desiccants in an amount to exhibit an Absorption Capacity (under 25° C.) of:
0.19 g per 10 ml volume of container under RH40%; and/or
0.27 g per 10 ml volume of container under RH80%
   should produce acceptable stability.

The above data shows that varenicline tartrate tablets meeting the U.S. Food and Drug Administration's (FDA) recommendations of not more than 18.5 ppm of nitrosamine impurities can be prepared by methods described herein including but not limited to the use of (i) dry blending/granulations, particularly with a low moisture excipient and/or (ii) seal coated tablets. The data further shows varenicline tartrate tablets can be prepared meeting the FDA's 18.5 ppm nitrosamine levels if the initial nitrosamine content of the bulk varenicline tartrate is below 5 ppm, preferably below 4 ppm and/or the total manufacturing time is less than 60 days, preferably 30 days. The data also demonstrated the addition of a desiccant to the packaging of varenicline tartrate tablets prepared in accordance with the present invention can keep the nitrosamine impurities below the FDA's 18.5 ppm nitrosamine limitation over time.

The following methods were used to test the drug and compositions described in above Examples:

### Related Substances Method

### 1. Related Substances Method for API

1.1 Method for Varenicline Tartrate Impurity-1 (VTI-1)

1.1.1 Chromatographic Conditions

| Parameters | Description | | |
|---|---|---|---|
| Column | Inertsil ODS-3 C18 | | |
| | 4.6 mm * 250 mm, 5 μm | | |
| Column temperature (° C.) | 30 | | |
| Injection volume (μL) | 10 | | |
| Wavelength (nm) | 238 | | |
| Flow rate (mL/min) | 1.0 | | |
| Mobile Phase A | 0.01 mol/L octane sulfonic acid | | |
| | sodium and 0.01 mol/L potassium | | |
| | dihydrogen phosphate solution (adjust | | |
| | pH by phosphoric acid to 2.50 ± 0.05) | | |
| Mobile Phase B | Acetonitrile | | |
| Mobile Phase gradient | Time (min) | A (%) | B (%) |
| | 0 | 79 | 21 |
| | 15 | 79 | 21 |
| | 35 | 50 | 50 |
| | 38 | 40 | 60 |
| | 43 | 40 | 60 |
| | 44 | 79 | 21 |
| | 55 | 79 | 21 |

1.1.2 Preparation of solution:
Mobile Phase A: Dissolve about 2.16 g of 1-octanesulfonic acid sodium salt and 1.36 g of Potassium dihydrogen phosphate in 1000 mL of water, mix well, adjust pH to 2.50±0.05 with phosphoric acid, filter, ultrasound and degas for 15 minutes.
Blank Solution (Diluent): Mobile Phase A:Mobile Phase B=79:21
Varenicline Tartrate Impurity-1 (VTI-1) (6, 10-methano-6H-pyrazino [2,3-h] [3] benzazepine, 7, 8 ,9, 10-tetrahydro-8-trifluoroacetyl) Stock Solution: Accurately weigh appropriate amount of VTI-1 reference standard into an amber volumetric flask and dissolve with proper amount of Diluent and dilute to the volume, shake well. Further dilute with Diluent to obtain a concentration of 50 μg/mL of VTI-1 stock solution.
Reference Solution: Pipet 1.0 mL of Test Solution into a 100-mL amber volumetric flask, dilute with Diluent to the volume and shake well. Pipet 1.0 mL of the above solution into a 10-mL amber volumetric flask, dilute with Diluent to the volume and shake well.
Test Solution: Accurately weigh about 25.0 mg of the Varenicline Tartrate sample into a 50-mL amber volumetric flask, dissolve with Diluent and dilute to the volume, shake well.

1.1.3 Total impurity=VTI-1 impurity+any unknown impurity above LOQ (0.05%)

1.2 Method for Varenicline Tartrate Impurity-2 (VTI-2)

1.2.1 Chromatographic Conditions

| Chromatographic system | High performance liquid chromatography | | |
|---|---|---|---|
| Chromatographic Column | Kromasil 100-5-C18 (4.6 × 250 mm, 5 μm) | | |
| Mobile phase | Mobile phase A: 0.01 mol/L | | |
| | octane sulfonic acid sodium | | |
| | and 0.01 mol/L monopotassium | | |
| | phosphate solution (adjust the pH to | | |
| | 2.50 ± 0.05 with phosphoric acid) | | |
| | Mobile phase B: Acetonitrile | | |
| Detection wavelength | 200 nm | | |
| Column temperature | 30° C. | | |
| Flow rate | 1.0 mL/min | | |
| Injection volume | 20 μL | | |
| Run time | 45 minutes | | |
| Mobile Phase gradient | | Mobile phase | Mobile phase |
| | Time (min.) | A % | B % |
| | 0 | 95 | 5 |
| | 8 | 95 | 5 |
| | 23 | 85 | 15 |
| | 28 | 40 | 60 |
| | 35 | 40 | 60 |
| | 35.1 | 95 | 5 |
| | 45 | 95 | 5 |

1.2.2 Preparation of solutions
Mobile Phase A: Weigh and transfer about 2.16 g of octane sulfonic acid sodium salt and 1.36 g of monopotassium phosphate into 1000 mL of ultrapure water, adjust the pH to 2.50±0.05 with phosphoric acid, filter with a microporous filter, ultrasonic degassing for 2 minutes. (Preparation of alternative volumes at the same concentrations is acceptable.).
Blank Solution (Diluent): Ultrapure water-Acetonitrile=95:5
Varenicline Tartrate Impurity-2 (VTI-2) (L(+)-tartaric acid monoethyl ester) Stock Solution: Accurately weigh about 45 mg of VTI-2 Reference Standard into a 10-mL volumetric flask, dissolve and dilute to the volume with acetonitrile and shake well.
Reference Solution: Accurately transfer 1.0 mL of VTI-2 Stock Solution into a 50-mL volumetric flask, dilute to the volume with Diluent, and shake well.
Test Solution: Accurately weigh about 300 mg of Varenicline Tartrate into a 10-mL volumetric flask, dissolve and dilute to the volume with Diluent and shake well. Prepare Test Solution in duplicate. (Protect from light after preparation).

### 2. Related Substances Method for Finish Product (Varenicline Tablet)

2.1 Method for Varenicline tablet

2.1.1 Chromatographic Conditions

| Parameter | Description | | |
|---|---|---|---|
| Column | waters XBridge Phenyl 3.5 μm 4.6 * 250 mm | | |
| Column temperature (° C.) | 40 | | |
| Injection volume (μL) | 30 | | |
| Wavelength (nm) | 238 | | |
| Flow rate (mL/min) | 1.0 | | |
| Mobile Phase A | 0.02 mol/L sulfuric acid − acetonitrile = 95:5 | | |
| Mobile Phase B | 0.02 mol/L sulfuric acid − acetonitrile = 75:25 | | |
| Gradient procedure | Time (min) | A (%) | B (%) |
| | 0 | 100 | 0 |
| | 25 | 100 | 0 |
| | 60 | 0 | 100 |
| | 65 | 0 | 100 |
| | 65.1 | 100 | 0 |
| | 75 | 100 | 0 |

2.1.2 Preparation of solution
Blank Solution: 0.1% phosphoric acid (adjust pH to 3.0 with strong ammonia solution)-acetonitrile 90:10. Dilute 1 mL of phosphoric acid with water to 1000 mL, adjust pH to 3.0 with strong ammonia solution. Add 100 mL of acetonitrile into 900 mL of the above solution, mix well.
Reference Stock Solution: Accurately weigh approximately 21.5 mg of Varenicline Tartrate Reference Standard (equal to 12.5 mg of Varenicline) into a 50-mL volumetric flask, dissolve and dilute with 0.1 N HCl to the volume, and shake well.
Reference Solution: Transfer 1.0 mL of Reference Stock Solution into a 25-mL volumetric flask, dissolve and dilute with the Blank Solution to the volume, and shake well. Transfer 1.0 mL of above solution into a 20-mL volumetric flask, dissolve and dilute with the Blank Solution to the volume, and shake well. (Solution preparation may be scale-up or scale-down as long as solution concentration is the same.)
Test Solution: Place two tablets of 1 mg strength or four tablets of 0.5 mg strength into a 20-mL volumetric flask, add 14 mL of Blank Solution, and shake for 30 minutes to disperse the

Varenicline Tablet, then sonicate for 30 minutes, dilute with Blank Solution to the volume, centrifuge in a centrifugal tube at 7500 rpm for 20 minutes, and take the supernatant as Test Solution.

### 3. Particle Size

3.1. Instrument

### Mastersizer 3000 Laser Particle Size Analyzer and Electronic Analytical Balance

3.2. Test conditions

| | | | Wet |
|---|---|---|---|
| Disperser | Hydro ev | Method | method |
| Injection mode | Pre dispersion | Light cover | 5%-12% |
| | injection | degree | |
| Particle type | Non spherical | Analysis | Common |
| | | model | model |
| Particle refractive index | 1.700 | Absorptivity | 0.1 |
| No of measurements | 3 | Density | 1.0 g/cm³ |
| Dispersant | N-hexane | Refractive | 1.390 |
| | solution of | index of | |
| | 0.1% span 85 | dispersant | |
| Background | 10 sec | Sample | 10 sec |
| measurement | | measurement | |
| holding time | | holding time | |
| Stirring speed | 2000 rpm | Analytical | Standard |
| | | sensitivity | |
| Ultrasonic dispersion | No | Analysis | Mastersizer |
| | | software | 3000 |
| Equilibrium time | 2 min | N/A | N/A |

3.3. Sample Preparation

3.3.1 Preparation N-hexane solution of 0.1% span 85.

3.3.2 Weigh about 0.04 g sample and add into 5 mL dispersant, and ultrasonic for 10 min.

3.4. Sample test

3.4.1 Cleaning instrument.

3.4.2 The above parameters are set,

3.4.3 Reconfirm the inspection parameters.

3.4.4 Background checks are required.

3.4.5 Samples tested and each for 3 times.

### 4. Nitrosamine Test Method for API and Finish Product

4.1. Instrument

UPLC-MS/MS, Thermo scientific; Orbitrap Exploris 120

4.2. Solution Preparations

4.2.1 Diluent: Methanol

4.2.2 Mobile phase A: Formic acid: Purified Water (0.1:100), transferred 500 mL purified water to 500 mL analysis bottle, then added 500 μL formic acid and well mixed. Ultrasonic Degassing for 5 min.

4.2.3 Mobile phase B: Formic acid: Methanol (0.1:100), transferred 500 mL methanol to 500 mL analysis bottle, then added 500 μL formic acid and well mixed. Ultrasonic degassing for 5 min.

4.2.4 Reference stock solution I: Weight about 2.0 mg of 8-nitroso-7,8,9,10-tetrahydro-6H-6,10-methanoazepino[4,5-g]quinoxaline (Nitro-P08) into a 100 mL volumetric flask. Dissolve with methanol and dilute to volume and well mixed (about 20 μg/mL of Nitro-P08).

4.2.5 Reference stock solution II: Accurately pipet 5.0 mL of reference stock solution I into a 200-mL volumetric flask, dilute with methanol to volume and well mixed (about 500 ng/mL of Nitro-P08).

4.2.6 Reference stock solution III: Accurately pipet 5.0 mL of reference stock solution II into a 50-mL volumetric flask, dilute with methanol to volume and well mixed (about 50 ng/mL of Nitro-P08).

4.2.7 Reference solution: Accurately pipet 1.0 mL of reference stock solution III into a 50-mL volumetric flask, dilute with methanol to volume and well mixed.

4.2.8 Sample Solution

4.2.8.1 for API

Weighted about 25 mg of sample into a 50 mL volumetric flask. Diluted to volume with Diluent and well mixed. Prepared 2 duplication in parallel.

4.2.8.2 for Finish Product

Grind 10 tablets for 1 mg strength or 20 tablets for 0.5 mg strength, accurately weigh and transfer about 0.61 g of powder (equivalent to about 5 mg of Varenicline Tartrate API) into a 50 mL stoppered test tube, add 10 mL of methanol, vortex for 1 min and treat with ultrasound for 20 min. Place it still for 10 min, take an appropriate amount of solution for centrifugation (10000 rpm, 10 min), and then take the supernatant as the test solution. Prepare in duplicates. (Note: Grind the tablets and weigh right before the test sample solution preparation.)

4.3. Instrument Method

| Parameters | Details | | |
|---|---|---|---|
| Column | Waters XSelect CSH Phenyl-Hexyl XP, | | |
| | 2.5 μm 130 Å, 150 × 4.6 mm) | | |
| Column temperature | 30° C. | | |
| Flow rate | 0.5 mL/min | | |
| Mobile phase A | 0.1% Formic acid aqueous solution | | |
| Mobile phase B | 0.1% Formic acid methanol solution | | |
| Gradient | Time (min) | A % | B % |
| | 0 | 70 | 30 |
| | 1.0 | 70 | 30 |
| | 6.0 | 20 | 80 |
| | 9.5 | 20 | 80 |
| | 10.0 | 0 | 100 |
| | 11.0 | 0 | 100 |
| | 11.1 | 70 | 30 |
| | 15.0 | 70 | 30 |

| | |
|---|---|
| Injection volume | 5 μL |
| Sample plate temperature | 8° C. |
| Ion source | ESI source, positive ion mode |
| Spray voltage | 3500 |
| Sheath flow (Arb) | 50 |
| Auxiliary gas (Arb) | 15 |
| Sweep Gas (Arb) | 0 |
| Vaporizer Temp (° C.) | 350° C. |
| Temperature in nebulizer | 350° C. |
| chamber | |
| Scan mode | PRM |
| Collision Energy (%) | 30 |
| Quantitative ion | 211.1105, 169.0762 |
| Resolution | 60000 |
| Switching time for MS | 7.5-11 min |

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. Varenicline or pharmaceutically acceptable salt thereof with less than 6 ppm of any varenicline nitrosamine impurity.

2. The varenicline or pharmaceutically acceptable salt thereof of claim 1 exhibits a particle size distribution (D50) of a range from 1 micron to 50 microns.

3. The varenicline or pharmaceutically acceptable salt thereof of claim 1 exhibits a particle size distribution (D90) less than 20 microns.

4. A solid varenicline oral dosage form with less than 20 ppm of any varenicline nitrosamine impurity.

5. The solid varenicline oral dosage form of claim 4 exhibits a moisture content of less than 7.5%.

6. The solid varenicline oral dosage form of claim 4 is packaged in a bottle packaging with a desiccant wherein the desiccant exhibits an absorption capacity at least 0.01 g or more, per 10 ml volume of the bottle.

7. The solid varenicline oral dosage form of claim 4 is packaged in a blister packaging wherein the blister forming material exhibits a water vapor transmission rate (WVTR) at least 0.05 g/m²*day.

8. The solid varenicline oral dosage form of claim 6 wherein the blister packaging is further placed in a bag along with a desiccant.

9. The solid varenicline oral dosage form of claim 4 comprises varenicline or pharmaceutically acceptable salt thereof and at least one low moisture excipient with a moisture content of less than 0.5%.

10. The solid varenicline oral dosage form of claim 9 comprises at least a coating layer.

11. A kit comprising a first bottle and a second bottle as described in claim 6 and printed administration instructions wherein the first bottle comprises eleven (11) tablets comprising 0.5 mg of varenicline or a pharmaceutically acceptable salt thereof and the second bottle comprises 7 to 200 tablets comprising 1.0 mg of varenicline or a pharmaceutically acceptable salt thereof and the printed instructions inform a user to (i) orally administer one (1) of the 0.5 mg tablets in the first bottle once a day for the first three days of treatment; (ii) to orally administer one (1) 0.5 mg tablet in the first bottle twice a day (B.I.D.) or every twelve hours on days 4 to 7 of treatment and (iii) to orally administer one (1) 1.0 mg tablet from the second bottle twice a day (B.I.D.) or every twelve hours beginning on day 8 of treatment until all the 1.0 mg tablets in the second bottle are administered.

12. The kit as described in claim 11 wherein the second bottle comprises 7 tablets.

13. The kit as described in claim 11 wherein the second bottle comprises 30 tablets.

14. The kit as described in claim 11 wherein the second bottle comprises 42 tablets.

15. The kit as described in claim 11 wherein the second bottle comprises 60 tablets.

16. The kit as described in claim 11 wherein the second bottle comprises 90 tablets.

17. The kit as described in claim 11 wherein the second bottle comprises 120 tablets.
